(19) United States
(12) Patent Application Publication
(10) Pub. No.: US 2012/0303131 A1
(43) Pub. Date: Nov. 29, 2012

(54) SURGICAL APPARATUS

(76) Inventor: Gursharan Singh Chana, Birmingham (GB)

(21) Appl. No.: 13/515,033

(22) PCT Filed: Dec. 9, 2010
(86) PCT No.: PCT/GB2010/052065

§ 371 Date: Jun. 11, 2012

Related U.S. Application Data
(60) Provisional application No. 61/285,850, filed on Dec. 11, 2009.

(30) Foreign Application Priority Data
Dec. 11, 2009 (GB) 0921719.1

(51) Int. Cl. A61B 17/90; A61F 2/38; A61B 17/16

(52) U.S. Cl. 623/20.32; 606/87; 606/88; 606/89; 606/80

(57) ABSTRACT

A single use cutting jig for use in cutting a bone in orthopaedic surgery, the cutting jig having: a first face shaped to receive the end of the bone to be cut, a second face provided with one or more cutting slot, the or each slot extending through the cutting jig from the second face to the first face and being shaped for receiving a cutting device, wherein the cutting jig is molded from plastic, and wherein the interior surface of the or each cutting slot is provided with a metal coating.

The present invention relates to surgical apparatus.

Surgery in relation to the resurfacing or replacement of joints of the human body following wear or damage is well known. In particular, hip replacement, knee replacement, ankle replacement and shoulder replacement may be mentioned. Such surgery involves not only prosthetic implants, but also a number of re-usable pieces of equipment, including trial implants, alignment jigs, cutting jigs and alignment rods. These are made of metal and are sterilised before re-use.

The present inventor has identified that there are problems associated with this setup.

It can prove less than straightforward to ensure that all biological matter is removed from the equipment before re-use, and there is considerable expense associated with the sterilisation, including costs involved with transport of the equipment to and from the sterilisation facilities. A surgical theatre must have sufficient sets of each piece of equipment such that there are always enough, even whilst used sets are away being sterilised. There must also be sufficient to ensure that a spare is to hand, should a given item become contaminated, e.g. through being accidentally dropped.

Additionally, a container filled with the equipment is heavy, causing health and safety issues with its lifting. A number of different sized pieces of equipment will be included, to take account of the fact that patients have different sized joints. As an example, there will be six different sized cutting jigs used for the femur.

It is also the case that, over time, use of the equipment leads to wear. This can be highly significant in the case of a jig for use in knee replacement, where high degrees of accuracy are required to ensure that the flexural and extension gaps are the correct size for good use of the replacement knee.

The present invention provides, in a first aspect, a single use cutting jig for use in cutting a bone in orthopaedic surgery, the cutting jig having:
a first face shaped to receive the end of the bone to be cut,
a second face provided with one or more cutting slot, the or each slot extending through the cutting jig from the second face to the first face and being shaped for receiving a cutting device,
wherein the cutting jig is molded from plastic, and
wherein the interior surface of the or each cutting slot is provided with a metal coating.

Such a cutting jig is beneficial in that it is lightweight as compared to metal jigs and can be disposed of after use rather than requiring sterilisation. Further, it avoids any problems of loss of accuracy due to repeated use, and thus wearing down, of the surfaces of the cutting slots.

By having metal coatings on the interior surfaces of the cutting slots, which are the surfaces that can be contacted by the cutting device, the problem of plastic debris being generated following contact of the cutting device with these surfaces is avoided. Clearly, this is important as the deposition of debris in the body of the patient during surgery should be avoided.

A benefit of the jig is that it can have a greater depth, i.e. the distance between the first face and the second face without being unduly heavy. This means that the cutting slots have a longer length, which in turn provides greater accuracy for cutting the bone in preparation for the implant.

In one embodiment, the depth of the jig is 15 mm or more, such as 20 mm or more, e.g. 25 mm or more. For example, the jig may have a depth of from 25 mm to 50 mm.

The cutting slots are suitably rectangular in cross section.

The cutting jig may also suitably be provided with one or more alignment holes on the second face, the or each hole extending through the cutting jig from the second face to the first face and being shaped for receiving an alignment rod. The surface of the or each alignment slot is provided with a metal coating. The alignment holes are suitably circular in cross section.

The cutting jig is suitably injection molded from plastic and therefore the moldable plastic used for the cutting jig is suitably a plastic that can be injection molded.

Preferably, the plastic is a recyclable plastic. This will then permit the cutting jig, once used, to be recycled. The metal coatings on the surfaces of the cutting slots and any alignment holes can readily be separated from the plastic material, thus leaving the plastic material to be recycled for use in non medical applications.

Examples of suitable plastics that can be used are polyaryletherketones (e.g. polyether ether ketones (PEEK)), polyphenylsulfones (e.g. Radel® PPSU), polyacetals and acetal copolymers (e.g. Pomalux®), nylons, polycarbonates, poly methyl methacrylates (PMMA). Other suitable medical grade plastics for instrumentation may also be used.

The metal that is used as the coating on the surfaces of the cutting slots and any alignment holes can be any metal suitable for use in medical applications. In this application references to metals include metal alloys. Suitable metals include stainless steel, alloys of nickel and titanium (e.g. nitinol) and titanium.

The metal coating can be applied to the surfaces of the cutting slots and any alignment holes by any suitable technique.

In one embodiment, the metal coating is applied by insert injection molding or flow injection molding.

In another embodiment, the metal coating is provided as a pre-formed metal component that can be attached to the jig so as to cover the required surface.

For example, the pre-formed metal component may be connected onto the jig in the required area by having corresponding male and female connecting components, such as lugs and correspondingly shaped holes, on the pre-formed metal component and the jig.

In another example, the pre-formed metal component may press fit into the plastic molded cutting jig at each end of the cutting slot.

As another alternative, the pre-formed metal component could be a simple box section with a protruding section (or “tooth”) at each end which would stop the component coming off the plastic jig.

The pre-formed metal component can fit into recesses at the start and end of the cutting slots such that the internal surface of the component lines up with the internal surface of the slot. This ensures that the cutting device, e.g. saw, does not get jammed when passing through the slot.

In one embodiment, the jig is a cutting jig for use in knee surgery or hip surgery. In one such embodiment, the jig is for use in knee surgery, for example it may be a cutting jig for use in preparing the femur.

The cutting jig may be for the left hand side of the body or the right hand side of the body. For example, it may be for a left knee or a right knee.

The invention also provides, in a second aspect, a single use alignment jig for use in creating alignment marks on a bone in orthopaedic surgery, the alignment jig having:
a first face shaped to receive the end of the bone to be cut,
a second face provided with one or more alignment drilling hole, the or each hole extending through the alignment jig from the second face to the first face and being shaped for receiving a drilling device,
wherein the alignment jig is molded from plastic, and
wherein the interior surface of the or each drilling hole is provided with a metal coating.

Such an alignment jig is beneficial in that it is lightweight as compared to metal jigs and can be disposed of after use rather than requiring sterilisation. Further, it avoids any problems of loss of accuracy due to repeated use, and thus wearing down, of the surfaces of the drilling holes.

By having metal coatings on the interior surfaces of the drilling holes, which are the surfaces that can be contacted by the drilling device, the problem of plastic debris being generated following contact of the drilling device with these surfaces is avoided. Clearly, this is important as the deposition of debris in the body of the patient during surgery should be avoided.

A benefit of the jig is that it can have a greater depth, i.e. the distance between the first face and the second face without being unduly heavy. This means that the drilling holes have a longer length, which in turn provides greater accuracy for drilling the alignment marks.

In one embodiment, the depth of the jig is 15 mm or more, such as 20 mm or more, e.g. 25 mm or more. For example, the jig may have a depth of from 25 mm to 50 mm.

The drilling holes are suitably circular in cross section.

The alignment jig is suitably injection molded from plastic and therefore the mouldable plastic used for the alignment jig is suitably a plastic that can be injection molded.

Preferably, the plastic is a recyclable plastic. This. will then permit the alignment jig, once used, to be recycled. The metal coatings on the surfaces of the drilling holes can readily be separated from the plastic material, thus leaving the plastic material to be recycled for use in non medical applications.

Examples of suitable plastics that can be used are as described above in relation to the first aspect.

The metal that is used as the coating on the surfaces of the drilling slots can be any metal that is suitable for use in medical applications. For example, it may be any of the metals as described above in relation to the first aspect.

The metal coating can be applied to the surfaces of the drilling holes by any suitable technique. Preferably, the metal coating is applied by insert injection molding or flow injection molding.

In one embodiment, the jig is an alignment jig for use in knee surgery or hip surgery. In one such embodiment, the jig is for use in knee surgery, for example it may be an alignment jig for use in preparing the femur.

The alignment jig may be for the left hand side of the body or the right hand side of the body. For example, it may be for a left knee or a right knee.

The invention also provides, in a third aspect, a kit for use in orthopaedic surgery, the kit comprising:
(a) a cutting jig as defined in the first aspect; and
(b) one or more cutting device.

It may be that the kit includes a cutting jig for the left side or the right side of the body. In one embodiment, the kit is for the left side only. In another embodiment, the kit is for the right side only. In another embodiment, the kit includes a cutting jig for the left side of the body and a cutting jig for the right side of the body.

The cutting device may suitably be a saw.

In one embodiment, the kit further comprises:
(c) one or more alignment rod.

The or each alignment rod may, in a preferred embodiment, be molded from plastic. The plastic may be a plastic as described above in relation to the first aspect. In one embodiment, the alignment rods are molded from the same plastic as the cutting jig.

In one embodiment, the kit further comprises:
(d) an alignment jig as defined in the second aspect.

When the kit includes a cutting jig for the left side of the body, the alignment jig is also for the left side of the body. When the kit includes a cutting jig for the right side of the body, the alignment jig is also for the right side of the body. When the kit includes a cutting jig for the left side of the body and a cutting jig for the right side of the body, the kit suitably includes an alignment jig for the left side of the body and an alignment jig for the right side of the body.

In one embodiment, the kit further comprises:
(e) one or more drilling device.

In one embodiment, the kit further comprises:
(f) one or more alignment pin.

The or each alignment pin may, in a preferred embodiment, be molded from plastic. The plastic may be a plastic as described above in relation to the first aspect.

The invention also provides, in a fourth aspect, a kit for use in orthopaedic surgery, the kit comprising:
(i) an alignment jig as defined in the second aspect; and
(ii) one or more drilling device.

It may be that the kit includes an alignment jig for the left side or the right side of the body. In one embodiment, the kit is for the left side only. In another embodiment, the kit is for the right side only. In another embodiment, the kit includes an alignment jig for the left side of the body and an alignment jig for the right side of the body.

In one embodiment, the kit further comprises:
(iii) one or more alignment pin.

In one embodiment, the kit further comprises:
(iv) a cutting jig as defined in the first aspect.

When the kit includes an alignment jig for the left side of the body, the cutting jig is also for the left side of the body. When the kit includes an alignment jig for the right side of the body, the cutting jig is also for the right side of the body. When the kit includes an alignment jig for the left side of the body and an alignment jig for the right side of the body, the kit suitably includes a cutting jig for the left side of the body and a cutting jig for the right side of the body.

In one embodiment, the kit further comprises:
(v) one or more cutting device, such as a saw.

In one embodiment, the kit further comprises:
(vi) one or more alignment rod.

The or each alignment rod may, in a preferred embodiment, be molded from plastic. The plastic may be a plastic as described above in relation to the first aspect.

The invention also provides, in a fifth aspect, a trial tibial implant kit, for use in determining the correct size of tibial implant for a patient during knee resurfacing or replacement surgery, the kit comprising:
(1) a trial tibial implant base unit, that has a proximal surface and a distal surface, and that has a medial portion for location over the medial tibial condyle of a tibia, and a lateral portion for location over the lateral tibial condyle of said tibia;
(2) one or more trial tibial extension unit, each unit having a proximal surface and a distal surface and each unit having a medial portion for location over the medial tibial condyle of a tibia, and a lateral portion for location over the lateral tibial condyle of said tibia;
wherein in the trial tibial implant base unit the proximal surface is shaped as the bearing surface of the tibia, which can engage and articulate with a femoral component, and the distal surface is shaped for releasably receiving and securing the proximal surface of a tibial tray or the proximal surface of a tibial tray extension unit;
wherein in the or each trial tibial extension unit the proximal surface is shaped for being received and releasably secured to the distal surface of the trial tibial tray base unit or the distal surface of another trial tibial extension unit and the distal surface is shaped for releasably receiving and securing the proximal surface of a tibial tray or the proximal surface of another trial tibial extension unit;
such that the base unit on its own, or when attached to one or more extension units, forms a trial tibial implant that has a medial portion for location over the medial tibial condyle of a tibia, and a lateral portion for location over the lateral tibial condyle of said tibia, and a proximal face that is shaped to engage and articulate with a femoral component, and a distal face, opposite the proximal face, shaped to fit with the proximal face of a tibial tray.

The trial tibial implant kit is beneficial because one or more tibial tray extension units can be built up on the base unit to create the correct size of trial tibial implant for a given patient. This means that various different sized trial tibial implants do not need to be used—and thus contaminated during the exercise of the surgeon establishing the correct size of implant. Rather, the surgeon can initially use the base unit on its own and then add one or more extension units until the correct size is achieved.

Preferably, the base unit and extension units are molded from plastic. Examples of suitable plastics are as described above in relation to the first aspect. If the units are made from plastic then they can be disposed of after a single use rather than needing to be sterilised. It also reduces the weight of any kit containing the units.

The kit may further comprise: a tibial tray that has a medial portion for location over the medial tibial condyle of a tibia, and a lateral portion for location over the lateral tibial condyle of said tibia, the tray having a proximal surface and a distal surface, the proximal surface being shaped for being received and releasably secured on the distal surface of the trial tibial implant base unit or a trial tibial extension unit and the distal surface being shaped for receiving and securing the proximal surface of the tibia.

Preferably, the tibial tray is molded from plastic. Examples of suitable plastics are as described above in relation to the first aspect.

In one preferred embodiment, the trial tibial implant base unit has a depth of 10 mm and therefore corresponds to the smallest size of tibial implant. The extension units suitably each have a depth of 2 mm. Therefore, for example, a kit with one base unit and three extension units allows tibial implant sizes of 10, 12, 14, and 16 mm to be assessed, whilst a kit with one base unit and five extension units allows tibial implant sizes of 10, 12, 14, 16, 18 and 20 mm to be assessed.

Therefore a preferred kit includes two or more, three or more, four or more, such as five, extension units.

In one embodiment, each extension unit is the same shape and size.

In another preferred embodiment, the trial tibial implant base unit has a depth of 10 mm and therefore corresponds to the smallest size of tibial implant, and there are three extension units with depths of 2 mm, 4 mm and 6 mm. This allows the base unit to be adjusted by amounts of 2 mm, 4 mm, 6 mm, 8 mm (2 mm+6 mm), 10 mm (4 mm+6 mm) and 12 mm (2 mm+4 mm+6 mm). Therefore a kit having a total of four units allows 10, 12, 14, 16, 18, 20 and 22 mm heights to be trialled. This reduces the inventory while increasing the range of adjustment.

In this embodiment, further extension units may be provided if this is desired, for example an extension unit with a depth of 8 mm could be provided or a second 2 mm depth extension unit could be provided.

In one embodiment, the proximal surface of the trial tibial implant base unit and the distal surface of the trial tibial extension unit are provided with corresponding attachment components, for example corresponding male and female attachment components, such as lugs and correspondingly shaped holes. Preferably, the male attachment components, e.g. lugs, are provided on the proximal surface of the trial tibial extension unit and the female attachment components, e.g. holes, are provided on the distal surface of the trial tibial implant base unit.

In one embodiment, the proximal surface of the trial tibial extension unit and the distal surface of the trial tibial extension unit are provided with corresponding attachment components, such that a first extension unit can be placed on and secured to a second such extension unit. For example, corresponding male and female attachment components, such as lugs and correspondingly shaped holes, may be used. Preferably, the male attachment components, e.g. lugs, are provided on the proximal surface of the trial tibial extension unit and the female attachment components, e.g. holes, are provided on the distal surface of the trial tibial extension unit.

There may be any suitable number of attachment components provided on each surface (apart from the proximal surface of the base unit which does not require attachment components) e.g. one, two, three, four, five or more male attachment components and the same number of corresponding female attachment components may be used on each surface.

Accordingly, in a preferred embodiment, the trial tibial implant base unit has a proximal surface that is shaped as the bearing surface of the tibia, which can engage and articulate with a femoral component, and a distal surface that is provided with holes for receiving correspondingly shaped lugs. The or each trial tibial extension unit has a proximal surface that is provided with lugs, and a distal surface that is provided with holes for receiving correspondingly shaped lugs.

There may be one or more lug on the proximal surface of each extension unit and one or more hole on the distal surface of the base unit and the distal surface of each extension unit. Preferably, there are two or more lugs on the proximal surface of each extension unit and two or more holes on the distal surface of the base unit and the distal surface of each extension unit.

In one embodiment, there are one, two, three, four or five lugs on the proximal surface of each extension unit and the same number of holes on the distal surface of the base unit and on the distal surface of each extension unit.

Preferably, there is one or more lug located on the medial portion and one or more lug located on the lateral portion of the proximal surface of each extension unit. Preferably there are correspondingly located holes on the distal surface of the base unit and on the distal surface of each extension unit.

In one embodiment, the holes on the base unit are the same size and are arranged in the same layout as the holes on the or each extension unit. This enables the lugs provided on the proximal surface of the extension unit(s) to fit into the holes on the distal surface of the base unit and to fit into the holes on the distal surface of another extension unit.

When present, the tibial tray may have one or more lugs provided on its proximal surface to fit with the holes on the distal surface of the base unit and to fit into the holes on the distal surface of an extension unit. Preferably, the tibial tray has the same number, size and layout of lugs as the or each extension unit.

The invention will be further described, by means of example only, by reference to the drawings in which:

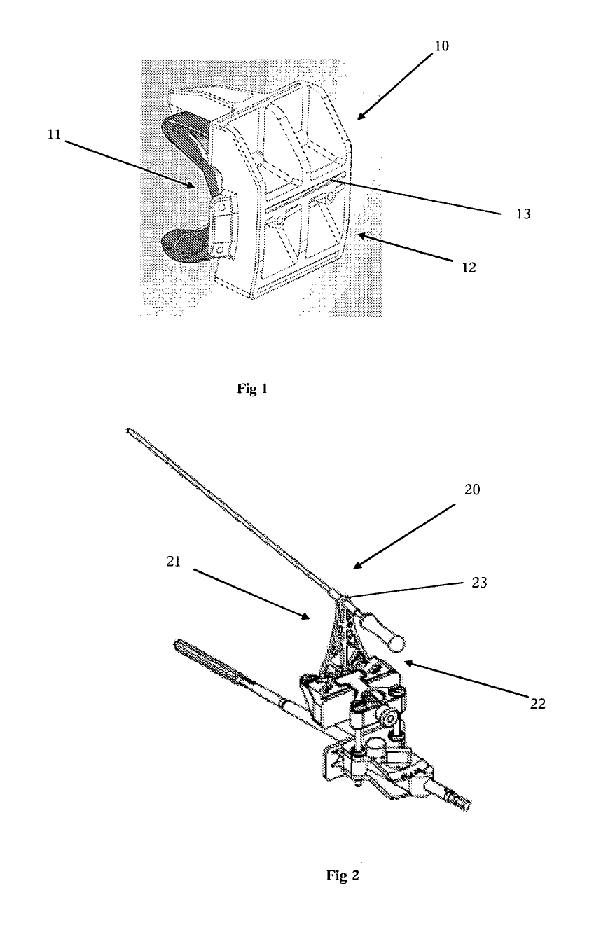

FIG. 1 shows a femoral cutting jig in accordance with the invention;

FIG. 2 shows a femoral alignment jig in accordance with the invention;

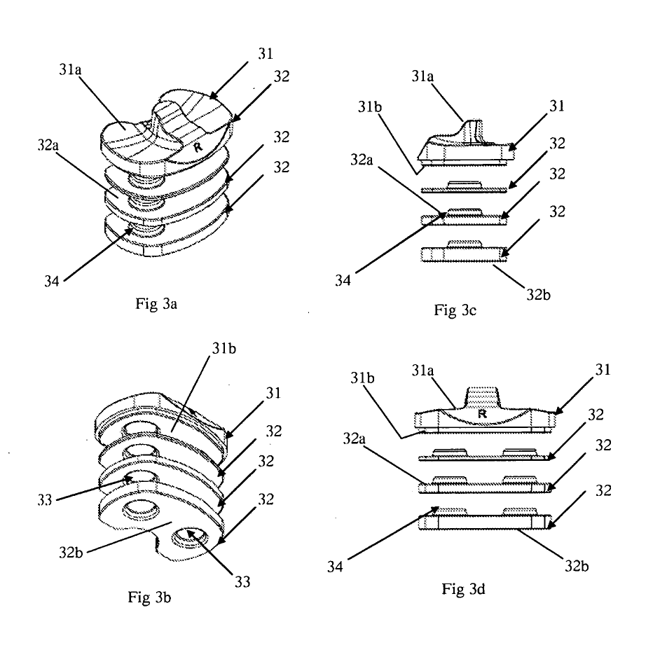

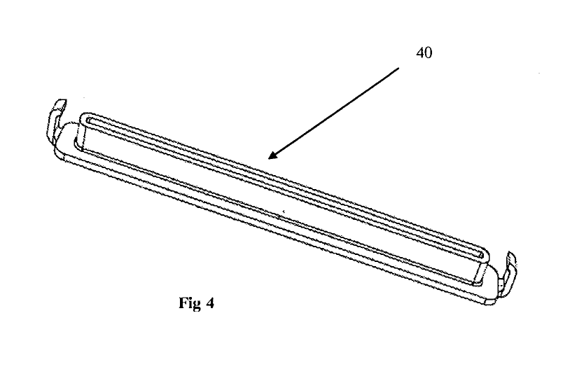

FIG. 3 shows a trial tibial tray kit in accordance with the invention; and

FIG. 4 shows a pre-formed metal component that can be used to provide the metal surface on the cutting slot of a cutting jig, such as the jig of FIG. 1.

FIG. 1 shows a single use femoral cutting jig 10 for use in cutting a femur in knee replacement surgery. The cutting jig has a first face 11 shaped to receive the end of the femur and a second face 12.

The second face 12 is provided with a cutting slot 13 extending through the cutting jig from the second face to the first face. The cutting slot is rectangular in cross section and is shaped for receiving a cutting saw.

The interior surface of the cutting slot 13 is provided with a metal coating, such as stainless steel.

The cutting jig is injection molded from medical grade plastic.

FIG. 2 shows a single use femoral alignment jig 20 for use in creating alignment marks on the femur in knee surgery.

The alignment jig 20 has a first face 21 shaped to contact the end of the bone to be cut and a second face 22.

The second face 22 is provided with an alignment drilling hole 23 that is circular in cross section. The hole 23 extends through the alignment jig from the second face to the first face and is shaped for receiving a drilling device.

The interior surface of the alignment hole 23 is provided with a metal coating, such as stainless steel.

The alignment jig is injection molded from medical grade plastic.

FIG. 3 shows a trial tibial implant kit, for use in determining the correct size of tibial implant for a patient during knee resurfacing or replacement surgery. The kit includes a trial tibial implant base unit 31 and a number of trial tibial extension units 32. In the kit shown there are three extension units 32 but the kit could include more or less of these extension units.

The trial tibial implant base unit 31 has a medial portion for location over the medial tibial condyle of a tibia, and a lateral portion for location over the lateral tibial condyle of said tibia. The base unit 31 has a proximal surface 31a and a distal surface 31b. The proximal surface 31a is shaped as the bearing surface of the tibia, which can engage and articulate with a femoral component. The distal surface 31b is provided with a number of holes 33. In the unit shown there are two holes 33, but the unit could have more or fewer of these holes.

One hole 33 is provided in the medial portion and one hole 33 is provided in the lateral portion.

The trial tibial extension units 32 also have a medial portion for location over the medial tibial condyle of a tibia, and a lateral portion for location over the lateral tibial condyle of said tibia. The extension units 32 have a proximal surface 32a and a distal surface 32b. The proximal surface 32a is provided with a number of lugs 34. In each of the extension units shown there are two lugs 34, but the unit could have more or fewer of these lugs.

One lug 34 is provided in the medial portion and the second lug 34 is provided in the lateral portion. The distal surface 32b is provided with a number of holes 33. In each extension unit shown there are two holes 33, but the units could have more or fewer of these holes. One hole 33 is provided in the medial portion and the other hole 33 is provided in the lateral portion.

The lugs 34 are shaped, sized and located so as to be received in the holes 33.

The base unit 31 on its own, or when attached to one or more extension units 32, forms a trial tibial implant that has a medial portion for location over the medial tibial condyle of a tibia, and a lateral portion for location over the lateral tibial condyle of said tibia, and a proximal face 31a that is shaped to engage and articulate with a femoral component, and a distal face 32b, opposite the proximal face, shaped to fit with the proximal face of a tibial tray.

FIG. 4 shows a pre-formed metal component 40 that can be used to provide the metal coating for the cutting slots of a cutting jig, such as that of FIG. 1.

The pre-formed metal component 40 can be press fitted onto the plastic molded cutting jig.

1. A single use cutting jig for use in cutting a bone in orthopaedic surgery, the cutting jig having:
a first face shaped to receive the end of the bone to be cut,
a second face provided with one or more cutting slot, the or each slot extending through the cutting jig from the second face to the first face and being shaped for receiving a cutting device,
wherein the cutting jig is molded from plastic, and
wherein the interior surface of the or each cutting slot is provided with a metal coating.

2. The jig of claim 1, wherein one or more alignment holes are provided on the second face, the or each hole extending through the cutting jig from the second face to the first face and being shaped for receiving an alignment rod, and the surface of the or each alignment slot being provided with a metal coating.

3. (canceled)

4. The jig of claim 1, wherein the jig is injection molded from a plastic selected from polyaryletherketones, polyphenylsulfones, polyacetals and acetal copolymers, nylons, polycarbonates, and polymethyl methacrylates.

5. The jig of claim 1, wherein the metal that is used as the coating is selected from stainless steel, alloys of nickel and titanium, and titanium.

6. The jig of claim 1, which is a jig for use in knee surgery or hip surgery.

7. A kit for use in orthopaedic surgery, the kit comprising:
(a) a cutting jig as defined in claim 1; and
(b) one or more cutting device.

8. The kit of claim 7, which comprises (a) a cutting jig for the left side of the body or (b) a cutting jig for the right side of the body or (c) a cutting jig for the left side of the body and a cutting jig for the right side of the body.

9. The kit of claim 7, wherein the kit further comprises:
(c) one or more alignment rod molded from plastic.

10. The kit of claim 7, wherein the kit further comprises:
(d) an alignment jig wherein the alignment jig is a single use alignment jig for use in creating alignment marks on a bone in orthopedic surgery, the alignment jig having:
a first face shaped to receive the end of the bone to be cut,
a second face provided with one or more alignment drilling hole, each of the one or more drilling hole extending through the alignment jig from the second face to the first face and being shaped for receiving a drilling device,
wherein the alignment jig is molded from plastic, and
wherein the interior surface of each of the one or more drilling hole is provided with a metal coating.

11. The kit of claim 10, wherein the kit further comprises:
(e) one or more drilling device; and/or
(f) one or more alignment pin molded from plastic.

12-20. (canceled)

21. A kit for use in orthopaedic surgery as defined in claim 7, further comprising: a trial tibial implant kit, for use in determining the correct size of tibial implant for a patient during knee resurfacing or replacement surgery, the kit comprising:
(1) a trial tibial implant base unit, that has a proximal surface and a distal surface, and that has a medial portion for location over the medial tibial condyle of a tibia, and a lateral portion for location over the lateral tibial condyle of said tibia;
(2) one or more trial tibial extension unit, each unit having a proximal surface and a distal surface and each unit having a medial portion for location over the medial tibial condyle of a tibia, and a lateral portion for location over the lateral tibial condyle of said tibia;
wherein in the trial tibial implant base unit the proximal surface is shaped as the bearing surface of the tibia, which can engage and articulate with a femoral component, and the distal surface is shaped for releasably receiving and securing the proximal surface of a tibial tray or the proximal surface of a tibial tray extension unit;
wherein in each trial tibial extension unit the proximal surface is shaped for being received and releasably secured to the distal surface of the trial tibial tray base unit or the distal surface of another trial tibial extension unit and the distal surface is shaped for releasably receiving and securing the proximal surface of a tibial tray or the proximal surface of another trial tibial extension unit;
such that the base unit on its own, or when attached to one or more extension units, forms a trial tibial implant that has a medial portion for location over the medial tibial condyle of a tibia, and a lateral portion for location over the lateral tibial condyle of said tibia, and a proximal face that is shaped to engage and articulate with a femoral component, and a distal face, opposite the proximal face, shaped to fit with the proximal face of a tibial tray.
